(19) United States
(12) Patent Application Publication
(10) Pub. No.: US 2022/0387132 A1
(43) Pub. Date: Dec. 8, 2022

(54) ARTICULATED ARM WITH HANDGRIP AND LOCKING AND UNLOCKING ACTUATOR FOR SUPPORT OF AN EXTENDED FLEXIBLE MEDICAL INSTRUMENT

(71) Applicant: ROBOCATH, Rouen (FR)

(72) Inventors: Bruno FOURNIER, Saint Ouen (FR); Philippe BENCTEUX, Saint Martin Du Vivier (FR)

(21) Appl. No.: 17/776,550

(22) PCT Filed: Nov. 4, 2020
(86) PCT No.: PCT/FR2020/051994

§ 371 Date: May 12, 2022

(30) Foreign Application Priority Data
Nov. 15, 2019 (FR) FR1912794

(51) Int. Cl. A61B 90/50; A61B 34/30; F16M 13/04

(52) U.S. Cl. CPC A61B 90/50; A61B 34/30; F16M 13/04; A61B 2090/5025; A61B 2017/00384

(57) ABSTRACT

An articulated arm for support of an extended flexible medical instrument includes: a segment rotationally mobile around an axis of rotation, where the mobile segment can either be rotationally locked or unlocked, a handgrip including: a distal holding part to be hand held, a proximal support part connecting the distal holding part to the remainder of the structure, and an actuator on the distal holding part locking and unlocking mobility of the segment in rotation around the axis. The locking and unlocking actuator extends over the main part of the length of the distal holding part such that: pressure of the user's hand griping the distal holding part directly creates contact with the actuator, sufficient for automatically unlocking mobility of the segment in rotation and release of the distal holding part terminates contact with the locking and unlocking actuator, sufficient for automatically locking mobility of the segment in rotation.

### CROSS-REFERENCE RELATED TO PRIORITY APPLICATIONS

This application is the U.S. national phase of International Application No. PCT/FR2020/051994 filed Nov. 4, 2020 which designated the U.S. and claims priority to FR 1912794 filed Nov. 15, 2019, the entire contents of each of which are hereby incorporated by reference.

### BACKGROUND OF THE INVENTION

### Field of the Invention

The invention relates to the field of articulated arms with handgrip and locking and unlocking actuator for support of an extended flexible medical instrument.

### Description of the Related Art

According to the prior art a catheter articulated arm is known having a handgrip with which the hand of the user removes this articulated arm, where this articulated arm is further attached to an operating table on which a patient is stretched out.

This articulated arm does not have a true system for rotational locking and unlocking of the articulated arm in a preferred position. In fact, this preferred position is only imperfectly stabilized with a permanent friction of various elements of the articulated arm against each other. A disadvantage is that an impact that is notable but not very large can change the chosen position of the articulated arm, and this could happen even during the operation on the patient.

### SUMMARY OF THE INVENTION

Another system could be imagined which would have the push button in the area of the handgrip. In that way, when the user would grip the handgrip in their hand, the user could with one of their fingers use it to next actuate an actuator for rotational locking and unlocking of the rotationally mobile segments of the articulated arm.

This other system would have the advantage of automatically unlocking the articulated arm when the hand of the user released the handgrip.

But there would still remain both:
[symbol] the disadvantage of having to make two distinct and independent movements for first griping the handgrip and then commanding the rotational locking of the various segments of the articulated arms,
[symbol] and also the disadvantage of providing either two different push buttons or two options for push-button arrangement, in order to correspond to the two different situations which are the holding of the handgrip by the left-hand of a left-handed user and the holding of the handgrip by the right hand of a right-handed user.

The goal of the present invention is to provide an articulated arm at least partially remedying the aforementioned disadvantages.

More specifically, the invention aims to provide both an articulated arm operating nearly equally well with the left-hand as with a right-hand of the user of this articulated arm, and also a locking and unlocking system which achieves a good compromise between automation, simplicity and effectiveness.

In that way, the articulated arm called for by the invention will operate equally well for holding of the handgrip of the articulated arm done with a left-hand as with a right-hand.

Further, the articulated arm called for by the invention will perform an automatic rotational locking of the at least one rotationally mobile segment (or even several or all of the rotationally mobile segments of the articulated arm simultaneously) during holding or griping of the handgrip of the articulated arm by the hand of the user equally well as an automatic rotational unlocking of the at least one rotationally mobile segment (or even several or all of the rotationally mobile segments of the articulated arm simultaneously) during the releasing or letting go of the articulated arm by the hand of this user who steps back.

For this purpose, the present invention proposes an articulated arm for support of an extended flexible medical instrument comprising: at least one segment rotationally mobile around an axis of rotation, where the mobile segment can either be rotationally locked around the axis of rotation or rotationally unlocked around the axis of rotation, a handgrip comprising: a distal holding part, with an elongated shape, intended to be held by the hand of the user of the articulated arm, a proximal support part connecting the distal holding part to the remainder of the structure of the articulated arm, and an actuator for locking and unlocking the mobility of the segment in rotation around the axis of rotation, disposed on the distal holding part, characterized in that the locking and unlocking actuator extends over the main part of the length of the distal holding part such that: a simple pressure of the hand of the user griping the distal holding part directly exerts a contact with the locking and unlocking actuator which is sufficient for automatically unlocking the mobility of the segment in rotation around the axis of rotation and the simple release of the distal holding part by the hand of the user directly leads to an absence of contact with the locking and unlocking actuator which is sufficient for automatically locking the mobility of the segment in rotation around the axis of rotation.

The articulated arm is advantageously robotic.

According this other system previously brought up, the use would proceed in the following way:
[symbol] in a first step, the user would take hold of the handgrip with one hand;
[symbol] in a second step, they would unlock the brake;
[symbol] in a third step, that would allow them to move the robot to the desired position, since the arm is designed to fluidly accompany the movements of the robot, in particular because of the internal cylinders which would cancel the weight of the robot born by the articulated arm;
[symbol] in a fourth step, they would again lock the brake;
[symbol] in a fifth step, there would let go of the handgrip.

According to the preferred embodiments of the invention, the articulated arm can be considered as merging, from a point of view of the user, both the first and second steps with each other and also the fourth and fifth steps. This would have the double advantage of making manipulation easier and making it impossible for the user to release the handgrip while forgetting to relock the brake, which otherwise would have the risk of the robot moving during the operation which could then cause an uncontrolled movement of the guides and catheters inside the patient, with potentially severe consequences for the health of this patient.

According to the preferred embodiments, the invention comprises one or more of the following characteristics which can be used separately or in combinations of some thereof or in combination of all thereof.

Preferably, said contact is a pressure mechanical contact.

Preferably, said locking and unlocking actuator is a press button: which, when it is directly or indirectly depressed, directly and mechanically actuates the closure, or respectively the opening, of an electrical contact located inside the distal holding part, during rotational unlocking of the segment, and which, when it is released, directly and mechanically actuates the opening, or respectively the closing, of this electrical contact, during rotational locking of the segment.

Preferably, said locking and unlocking actuator comprises at least two press buttons: depressing these two buttons directly and mechanically actuates the closure, or respectively the opening, of an electrical contact located inside the distal holding part, during rotational unlocking of the segment, and releasing at least one of these two press buttons directly and mechanically actuates the opening, or respectively the closing, of this electrical contact, during rotational locking of the segment.

Preferably, while it is depressed, the press button slides in one or more guiding rails, or while they are depressed, the press buttons slide in one or more guiding rails.

Thus, the simple mechanical pressure exerted by the hand of the user who grips the handgrip of the mechanical arm is sufficient for directly and automatically actuating the rotational locking and unlocking actuator, here in unlocked mode. Similarly, the release of this simple mechanical pressure exerted by the hand of the user who releases or lets go of the handgrip of the mechanical arm is sufficient for directly and automatically actuating the rotational locking and unlocking actuator, here in locked mode.

While mechanical pressure is exerted by the hand of the user on a single press button, ergonomics is preferred to the extent that the holding and releasing of this single press button is very fluid. While mechanical pressure is exerted by the hand of the user on at least two press buttons, security is preferred, to the extent where holding of both pressing buttons is necessary for releasing the mobility of the articulated arm for support of an extended flexible medical instrument whereas the release of one of these buttons is required for again blocking the mobility of the articulated arm for support of an extended flexible medical instrument.

Preferably, said contact is a capacitive electrical contact.

Preferably, the outer surface of the distal holding part comprises at least two capacitive zones: a simultaneous contact of these two capacitive zones by a single hand of the user automatically unlocks the mobility of the segment in rotation around the axis of rotation, and an absence of simultaneous contact with these two capacitive zones by a single hand of a user automatically locks the mobility of the segment in rotation around the axis of rotation.

Thus, the single mechanical pressure is replaced by a single touch contact with the hand of the user on the handgrip of the articulated arm.

Preferably the distal holding part is straight.

That way, the structure of the handgrip is simpler. In the case where the handgrip is mobile, movement thereof is also simpler.

Preferably, the proximal support part has an elongate shape, preferably straight, and the distal holding part is longer than the proximal support part.

That way, the structure of the handgrip is simpler. The dimension of the handgrip is also reduced.

Preferably, the distal holding part and the proximal support part are orthogonal to each other.

Preferably, the distal holding part has a cylindrical shape, advantageously with a circular section.

Thus, the holding of the handgrip again leads more directly and more automatically to the actuation of the rotational locking and unlocking actuator.

Preferably, the junction between the rotational locking and unlocking actuator on the one hand and the distal holding part on the other is a sealed junction which remains sealed even during actuation of said actuator.

Thus, the seal of the handgrip of the articulated arm is provided both at rest and also in cleaning phase of the articulated arm. The seal of the handgrip of the articulated arm during operation on the patient is for its part additionally provided by a supplemental device in the form of a sterile barrier between the consumable portion and the nonconsumable portion of the articulated arm of the medical operation robot.

Preferably, said sealed junction comprises an O-ring arranged around the rotational locking and unlocking actuator, and sliding in the body of the distal holding part if said actuator is mobile.

Thus this seal joint shape has a particularly simple structure.

Preferably, said sealed junction comprises a bellows arranged around the rotational locking and unlocking actuator, and compressing and extending during movement of said mobile actuator.

In that way, this sealed joint shape provides a greater amplitude of range of movement of the rotational locking and unlocking actuator during actuation thereof.

Preferably said sealed junction comprises a sleeve covering the rotational locking and unlocking actuator and surrounding at least the majority of the distal holding part, where this sleeve is sufficiently deformable that the pressure from the hand of the user of the articulated arm actuates the rotational locking and unlocking actuator.

Thus this seal joint shape has a particularly robust and impermeable structure.

Preferably, the articulated arm comprises a system of cylinders internal to the articulated arm canceling the weight of the articulated arm for a hand of a user who moves the articulated arm after having held the handgrip of the articulated arm.

In that way, this assistance in the movement of the articulated arm, also contributes, as does the direct and automatic actuation of the rotational locking and unlocking actuator during the holding of the handgrip of the articulated arm by the hand of the user, to improving the fluidity of handling the articulated arm by the user.

Preferably, the articulated arm comprises several segments mobile in rotation around one or several axes of rotation each mobile segment of which can be either rotationally locked around the axis of rotation thereof or rotationally unlocked around the axis of rotation thereof, and the locking and unlocking actuator commands the locking and unlocking of the rotational mobility of several of said segments, preferably rotationally locking and unlocking the mobility of all said segments.

Thus, since unlocking, which provides fluidity of rotational movement of the articulated arm (when the one or more segments move), and locking, which provides effective rotational blocking (when the one or more segments are immobile), is shared by several rotationally mobile segments, even all the rotationally mobile segments, this makes the rotational unlocking and rotational locking command so much simpler, more practical and even more ergonomic for the user of the articulated arm.

Preferably, the articulated arm comprises at least three segments rotationally articulated relative to each other, around a single rotational direction, among which: a proximal segment located closest to the operating table when the articulated arm is attached onto this operating table, a distal segment, which carries the extended flexible medical instrument, and an intermediate segment located between the proximal segment and the distal segment.

Thus, the unlocking, which assures the fluidity of rotational movement of the articulated arm (when the one or more segments move), and the locking, which assures the effectiveness of the rotational blocking (when the one or more segments are immobile), are that much more significant when the structure of the articulated arm is complex and long.

Preferably, the extended flexible medical instrument comprises a catheter and/or a catheter guide and/or a guide catheter.

Other features and advantages of the invention will be apparent to the reader of the following description of a preferred embodiment of the invention, given as an example and with reference to the attached drawings.

### BRIEF DESCRIPTION OF THE DRAWINGS

FIG. 1 shows schematically in side view an example of an articulated arm incorporating a handgrip, and a locking and unlocking actuator according to an embodiment of the invention.

FIG. 2 shows schematically in top view an example of an articulated arm incorporating a handgrip, and a locking and unlocking actuator according to an embodiment of the invention.

FIG. 3 shows schematically in top view an example of a handgrip and a locking and unlocking actuator according to an embodiment of the invention.

FIG. 4 shows schematically in top view an example of internal structure of a handgrip and a locking and unlocking actuator according to an embodiment of the invention.

FIG. 5 shows schematically in side view example of seal joints for a handgrip and a locking and unlocking actuator according to an embodiment of the invention.

FIG. 6 shows schematically in side view example of seal joints for a handgrip and a locking and unlocking actuator according to an embodiment of the invention.

FIG. 7 shows schematically in side view example of seal joints for a handgrip and a locking and unlocking actuator according to an embodiment of the invention.

FIG. 8 shows schematically in top view an example of internal structure of a handgrip and a locking and unlocking actuator according to another embodiment of the invention.

FIG. 9 shows schematically in top view an example of internal structure of a handgrip and a locking and unlocking actuator according to yet another embodiment of the invention.

### DESCRIPTION OF THE PREFERRED EMBODIMENTS

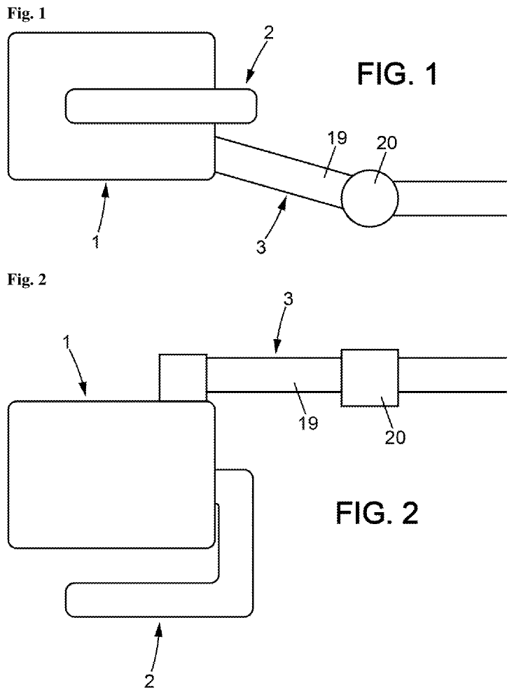

FIG. 1 shows schematically in side view an example of an articulated arm incorporating a handgrip, and a locking and unlocking actuator according to an embodiment of the invention.

FIG. 2 shows schematically in top view an example of an articulated arm incorporating a handgrip, and a locking and unlocking actuator according to an embodiment of the invention.

An articulated arm 3 comprises at least one segment 19 rotationally mobile around an axis of rotation 20. This articulated arm 3 comprises a robot 1 carrying the extended flexible medical instrument intended to be inserted into the patient and also a handgrip 2 for manipulation of the articulated arm 3 in the robot 1 carried by this articulated arm 3.

The user proceeds as follows:
[symbol] first, they take hold of the handgrip 2 with a hand, in fact with only one hand, left or right, as they prefer, according to whether they are left- or right-handed;
   ○ the simple fact of taking hold of the handgrip 2 automatically unlocks the brake;
   ○ the segment 19 is thus freely mobile in rotation around the axis of rotation 20;
[symbol] next, with the handgrip 2 firmly in hand, the user moves the robot 1 as they wish to the intended position, the articulated arm 3 then fluidly accompanying the movements of the robot 1, in particular because of the internal cylinders (not shown for reasons of clarity) which cancel the weight of the robot 1 born by the articulated arm 3;
[symbol] finally, they release the handgrip 2;
   ○ the simple fact of releasing the handgrip 2 again automatically locks the brake;
   ○ the segment 19 is thus blocked and locked in rotation around the axis of rotation 20.

The brake is an electrically controlled braking system which is actuated by the holding handgrip 2. This brake actuated by gripping the holding handgrip 2 can control one or more rotationally mobile segments, or even all the segments that are rotationally mobile and belong to the articulated arm 3.

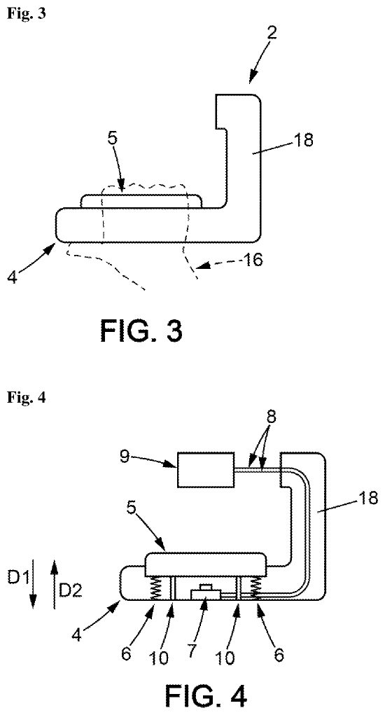

FIG. 3 shows schematically in top view an example of a handgrip and a locking and unlocking actuator according to an embodiment of the invention.

The handgrip 2 comprises a distal holding part 4 and a proximal part 18 for support of the distal part 4. The distal part 4 is intended to be gripped or taken hold of by the hand 16 of the user of the robot 1 carried by the articulated arm 3. The proximal part 18 connects the distal part 4 to the rest of the articulated arm 3. The distal part 4 has an extended shape, preferably cylindrical, with a section that is advantageously circular. The distal holding part 4 is advantageously at least two times longer than the proximal part 18 supporting this distal part 4.

A large press button 5 which, when depressed and released, commands respectively the unlocking and locking of the mobile segment 19 in rotation around the axis of rotation 20 extends over the length of distal part 4 extends over most of the length of this distal part 4, even over at least three quarters of the length of this distal part 4, even again more practically over the full length of this distal part 4. The length of this distal part 4 is along the horizontal direction in FIG. 3. When the hand of the user 16 grips the distal part 4 of the handgrip 2, the fingers of the hand 16 exert a mechanical pressure on the press button 5 which is then depressed into the distal part 4. When the hand of the user 16 releases the distal part 4 of the handgrip 2, the fingers of the hand 16 release the mechanical pressure on the press button 5 which then returns from the distal part 4.

The press button 5 is sufficiently large, meaning that it extends over a sufficient length of the distal part 4 of the handgrip 2 such that, when the hand 16 of the user grips this distal part 4, then this hand 16 of the user necessarily presses on the press button 5. This press button 5 is sufficiently large for operating with the right hand or the left hand of the user.

FIG. 4 shows schematically in top view an example of an internal structure of a handgrip and a locking and unlocking actuator according to an embodiment of the invention.

The distal part 4 of the handgrip comprises the press button 5. The internal structure of this distal part 4 also comprises a pair of compression springs 6, a push button 7, electric wires 8, an electronic card 9 for driving the braking system of the mobile segment 19 relative to the axis of rotation 20, and a pair of guide rails 10.

The press button 5 is a rigid part mounted on two compression springs 6 slidably in the two guiding rails 10 and whose pressing on the push button 7 causes the closure of an electrical contact. The two electrical wires 8 of the push-button 7 are connected to the electronic card 9 which is consequently able to drive the electrical command of the braking system.

When the hand of the user grips the distal holding part 4, the large press button 5 depresses into the body of this distal part 4 by sliding along the guiding rails 10, along the direction D1, by compressing the springs 6 for finally coming to bear, at the end of the translation range, on the push button 7 which then establishes an electronic contact with the driving electronic card 9 through the electric wires 8. The driving electronic card 9 triggers, starting with establishing this electrical contact, the blocking and locking of the brake(s) thus blocking the rotation of the mobile segment(s) 19 around the axis (axes) of rotation 20.

When the hand of the user releases the distal holding part 4, the large press button 5 returns from the body of this distal part 4 again sliding along the guiding rails 10, along the direction D2, by being pushed back by the springs 6 during decompression, for finally coming to release the push button 7 which then brakes the electronic contact previously established with the driving electronic card 9 through the electric wires 8. The driving electronic card 9 triggers, starting with braking this electrical contact, the unblocking and unlocking of the brake(s) thus releasing the rotation of the mobile segment(s) 19 around the axis (axes) of rotation 20.

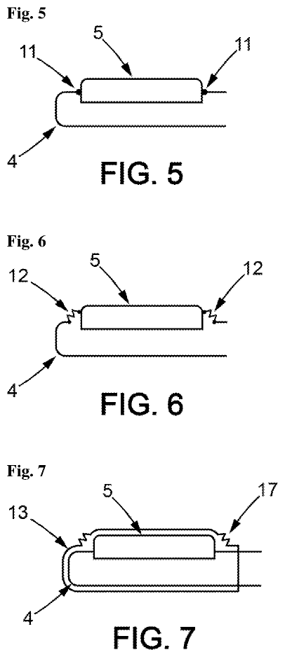

FIGS. 5 to 7 show schematically in side view examples of seal joints for a handgrip and a locking and unlocking actuator according to several embodiments of the invention.

The junction, between the press button 5 and the remainder of the body of the distal holding part 4 of the handgrip 2 is sealed. That way, this junction is going to be able to resist disinfection using liquid products at the end of the operation on the patient; during the operation on the patient, the seal against liquids in that case is provided by a skirt not shown here and which is independent of this invention.

In FIG. 5, the seal is provided by a joint 11 which can be either be an O-ring or a lip joint and which is located at the interface between the press button 5 and the remainder of the body of the distal holding part 4. This joint 11 goes around the press button 5 in a plane orthogonal to the plane of FIG. 5. The press button 5 is depressed into and returns from the remainder of the body of the distal holding part 4 by rubbing on the inside of the ring formed by the joint 11.

In FIG. 6, the seal is provided by a bellows 12. This bellows 12 is compressed when the press button 5 is depressed into the remainder of the body of the distal holding part 4. This bellows 12 is released when the press button 5 returns from the remainder of the body of the distal holding part 4.

In FIG. 7, the seal is provided by a flexible sleeve 13 which surrounds the assembly formed by both the press button 5 and also by the distal holding part 4 of the handgrip 2 or at least most of this distal part 4. This flexible sleeve 13 moves with the deformation related to pressing on the press button 5 by the hand of the user, for example either by the elasticity of the material from which it is made, or by a bellows shape 17 in the area of the interfaces located between both the press button 5 and also the distal holding part 4.

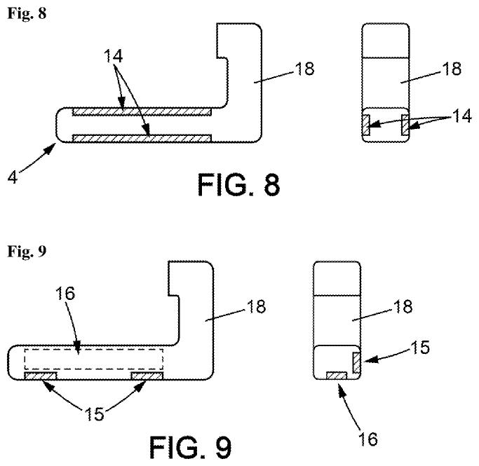

FIG. 8 shows schematically in top view an example of internal structure of a handgrip and a locking and unlocking actuator according to an embodiment of the invention.

The press button 5 from the preceding figures was replaced here by a single capacitive sensor 14 or capacitive zone 14. It is the simple contact with the hand of the user which is detected and which serves for activation of the locking and unlocking command. With concern for additional safety, two capacitive sensors 14 are used here, with the requirement that the hand of the user activate both capacitive sensors 14 simultaneously in order to cause the unlocking of the brake, meaning the activation of the unlocking command. The two capacitive sensors 14 are arranged on opposite sides or on opposite surfaces of the body of the distal holding part 4. The distal holding part 4 of the handgrip 2 advantageously has a cylindrical shape with square cross-section and rounded angles.

FIG. 9 shows schematically in top view an example of internal structure of a handgrip and a locking and unlocking actuator according to an embodiment of the invention.

The principal used is, as in FIG. 8, the principle of the use of one or more capacitive sensors. Increasing the number of capacitive sensors used serves to produce a better discrimination between an accidental press and an intentional press. In FIG. 9, both one of the two short capacitive sensors 15 and also the large capacitive sensor 16 have to be activated simultaneously, for example, in order to rotationally unlock the mobile segment(s) 19 in rotation around the axis (axes) of rotation 20. The short capacitive sensors 15 correspond to the placement of the thumb; third two short capacitive sensors 15, respectively for the right thumb and the left thumb, at the choice of the user according to whether they are right-handed or left-handed, for example. The large capacitive sensor 16 for its part corresponds to the end of the other four fingers of the hand of the user. The two short capacitive sensors 15 are shorter than the large capacitive sensor 16 which is larger than them. The two short capacitive sensors 15 are located on the same side of the body of the distal holding part 4 of the handgrip 2, advantageously near the two ends of this extended distal holding part 4. The large capacitive sensor 16 is located on a contiguous side or on a contiguous surface of the side or of the surface with the two short capacitive sensors 15, where the two short capacitive sensors 15 are located near the two ends of the large capacitive sensor 16. The grasping distal part 4 of the handgrip 2 advantageously has a cylindrical shape with square cross-section and rounded corners.

Of course, the present invention is not limited to the examples and the embodiment described and shown, but it could have many variants accessible to the person skilled in the art.

1. An articulated arm for support of an extended flexible medical instrument comprising:
at least one segment rotationally mobile around an axis of rotation, where the mobile segment can either be rotationally locked around the axis of rotation or rotationally unlocked around the axis of rotation,
a handgrip comprising:
a distal holding part, with an elongated shape, intended to be held by the hand of the user of the articulated arm,
a proximal support part connecting the distal holding part to the remainder of the structure of the articulated arm, and
an actuator for locking and unlocking the mobility of the segment in rotation around the axis of rotation, disposed on the distal holding part,
wherein the locking and unlocking actuator extends over the main part of the length of the distal holding part such that:
a simple pressure of the hand of the user griping the distal holding part directly exerts a contact with the locking and unlocking actuator which is sufficient for automatically unlocking the mobility of the segment in rotation around the axis of rotation, and
a simple release of the distal holding part by the hand of the user directly leads to an absence of contact with the locking and unlocking actuator which is sufficient for automatically locking the mobility of the segment in rotation around the rotational axis.

2. The articulated arm according to claim 1, wherein said contact is a pressure mechanical contact.

3. The articulated arm according to claim 2, wherein:
said locking and unlocking actuator is a press button:
which, when the press button is directly or indirectly depressed, directly and mechanically actuates the closure, or respectively the opening, of an electrical contact located inside the distal holding part, during rotational unlocking of the segment, and
which, when the press button is released, directly and mechanically actuates the opening, or respectively the closing, of this electrical contact, during rotational locking of the segment.

4. The articulated arm according to claim 2, wherein:
said locking and unlocking actuator comprises at least two press buttons:
depressing these two buttons directly and mechanically actuates the closure, or respectively the opening, of an electrical contact located inside the distal holding part, during rotational unlocking of the segment, and
releasing at least one of these two press buttons directly and mechanically actuates the opening, or respectively the closing, of this electrical contact, during rotational locking of the segment.

5. The articulated arm according to claim 3, wherein, while depressed, the press button slides in one or more guiding rails, or wherein while depressed, the press buttons slide in one or more guiding rails.

6. The articulated arm according to claim 1, wherein said contact is a capacitive contact.

7. The articulated arm according to claim 6, wherein:
the outer surface of the distal holding part comprises at least two capacitive zones:
a simultaneous contact of these two capacitive zones by a single hand of the user automatically unlocks the mobility of the segment in rotation around the axis of rotation, and
an absence of simultaneous contact with these two capacitive zones by a single hand of a user automatically locks the mobility of the segment in rotation around the axis of rotation.

8. The articulated arm according to claim 1, wherein the distal holding part is straight.

9. The articulated arm according to claim 1, wherein:
the proximal support part has an elongate shape,
and the distal holding part is longer than the proximal support part.

10. The articulated arm according to claim 9, wherein the distal holding part and the proximal support part are orthogonal to each other.

11. The articulated arm according to claim 1, wherein the distal holding part has a cylindrical shape.

12. The articulated arm according to claim 1, wherein the junction between the rotational locking and unlocking actuator and the distal holding part is a sealed junction which remains sealed even during actuation of said actuator.

13. The articulated arm according to claim 12, wherein said sealed junction comprises an O-ring arranged around the rotational locking and unlocking actuator, and sliding in the body of the distal holding part if said actuator is mobile.

14. The articulated arm according to claim 12, wherein said sealed junction comprises a bellows arranged around the rotational locking and unlocking actuator, and compressing and extending during movement of said mobile actuator.

15. The articulated arm according to claim 12, wherein said sealed junction comprises a sleeve covering the rotational locking and unlocking actuator and surrounding at least the majority of the distal holding part, where this sleeve is sufficiently deformable that the pressure from the hand of the user of the articulated arm actuates the rotational locking and unlocking actuator.

16. The articulated arm according to claim 1, further comprising a system of cylinders internal to the articulated arm canceling the weight of the articulated arm for a hand of a user who moves the articulated arm after having held the handgrip of the articulated arm.

17. The articulated arm according to claim 1, wherein:
the articulated arm comprises several segments mobile in rotation around one or several axes of rotation each mobile segment of which can be either rotationally locked around the axis of rotation thereof or rotationally unlocked around the axis of rotation thereof,
and wherein:
the locking and unlocking actuator commands the locking and unlocking of the rotational mobility of several of said segments.

18. The articulated arm according to claim 1, further comprising:
at least three segments rotationally articulated relative to each other, around a single rotational direction, among which:
a proximal segment located closest to the operating table when the articulated arm is attached onto this operating table,
a distal segment, which carries the extended flexible medical instrument, and an intermediate segment located between the proximal segment and the distal segment.

19. The articulated arm according to claim 1, wherein the extended flexible medical instrument comprises a catheter and/or a catheter guide and/or a guide catheter.

20. The articulated arm according to claim 1, wherein the distal holding part has a cylindrical shape with a circular section.
